(19) United States
(12) Patent Application Publication
(10) Pub. No.: US 2022/0183943 A1
(43) Pub. Date: Jun. 16, 2022

(54) PROCESS FOR PRODUCING SYNTHETIC SQUALANE AND SQUALANE DERIVATIVES

(71) Applicant: ADVONEX INTERNATIONAL CORP., Brockville (CA)

(72) Inventors: Chandrashekhar JOSHI, Kingston (CA); Sunhwa LEE, Brockville (CA); Dzmitry MALEVICH, Kingston (CA)

(21) Appl. No.: 17/604,198

(22) PCT Filed: Apr. 30, 2020
(86) PCT No.: PCT/CA2020/050574

§ 371 Date: Oct. 15, 2021

Related U.S. Application Data
(60) Provisional application No. 62/842,128, filed on May 2, 2019.

(51) Int. Cl. A61K 8/31; A61Q 19/00

(52) U.S. Cl. CPC A61K 8/31; A61Q 19/00

(57) ABSTRACT

The present application provides a process for synthesizing squalane and squalane derivatives as alternatives for squalene extracted from natural products. In particular, this application provides a process for producing a cosmetic, personal care or pharmaceutical composition comprising the steps of: (a) combining a Cn-fatty acid and a Cm-fatty acid with a solvent to form an electrolysis reaction mixture, wherein the sum of n+m is 32; and performing a Kolbe electrolysis on the electrolysis reaction mixture; (b) subjecting the product of step (a) to a hydroisomerization reaction to produce a C30 saturated, branched hydrocarbon or a mixture of C30 saturated, branched hydrocarbons; and (c) formulating the C30 saturated, branched hydrocarbon with one or more ingredients to produce the cosmetic, personal care or pharmaceutical composition. Also provided are cosmetic, personal care pharmaceutical compositions that comprise squalane and/or squalane derivatives produced by this process.

### CROSS-REFERENCE TO RELATED APPLICATIONS

The present application claims priority from U.S. Provisional Patent Application No. 62/842,128, filed May 2, 2019, which is incorporated herein in its entirety.

### FIELD OF THE INVENTION

The present application pertains to the field of processes for the synthesis of long chained hydrocarbons. More particularly, the present application relates to processes for the synthesis of long chained hydrocarbons for use in cosmetic and pharmaceutical formulations, and formulations made thereby.

INTRODUCTION

Squalane is a high-end moisturizing agent often found in personal care products, especially cosmetics. Squalane acts as a lubricating agent in the manufacture of skin care products, and when incorporated in skin care products it helps provide smooth and soft appearance to the skin. Squalane is also used in hair conditioning products. In addition to personal care products, squalane is also employed in the manufacture of vaccines.

Squalane has a molecular formula C₃₀H₆₂ and has the chemical structure shown below.

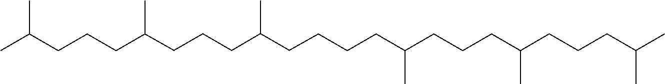 [SMILES: CC(C)CCCC(C)CCCC(C)CCCCC(C)CCCC(C)CCCC(C)C]

Commercially available squalane is extracted from olive oil or shark livers, or it is synthesized from sugar cane. To produce about a ton of squalane it is necessary to make use livers from approximately 3,000 sharks. Given the current global demand for nearly 2,500 tons of squalane per annum, this translates to killing of approximately 6 million deep-water sharks each year. Several of the shark species used for the production of squalane are endangered and, consequently, sea shark harvesting is now forbidden in most parts of the world. Furthermore, companies such as Unilever and L'Oreal have declared they will no longer procure shark liver squalane for their cosmetic products and are instead making use of plant-based sources for squalane. Since shark liver has been the second largest source of squalane (after olive oil), these changes are resulting in a significant reduction in the supply of squalane.

Olive oil-based squalane was the dominant segment of the squalane market in 2015. Olive oil-based squalane is made from hydrogenation of squalene that is extracted from the olive oil. Considering the composition, olive oil from the first extraction holds approximately 400 mg to 450 mg of squalene per 100g, while refined olive oil contains about 25% less. The finest quality olive oil may contain concentrations of nearly 700 mg of squalene per 100 g.

To address the global demand for squalane from olive oil requires 500-600,000 tons of olive oil, which corresponds to about 20-25% of global production. However, recently xylella fastidiosa bacteria has been devastating the Italian olive oil production with no known cure except for destruction of the diseased trees. Xylella has recently spread to the even larger Spanish olive industry and is threatening the Greek grooves resulting to overall reduction in global olive oil production. This is making it harder to obtain squalane from olive oil and alternatives are being sought that are less expensive.

An example of an alternative approach is being taken by Amyris, which makes a squalane through dimerization/hydrogenation of farnesene, a molecule derived from cane sugar.

Commercial application of squalane is being restrained by price volatility, owing to the inconsistent supply.

A need remains for a cost-effective alternative for producing squalane that does not rely on the use of shark liver or olive oil.

The above information is provided for the purpose of making known information believed by the applicant to be of possible relevance to the present invention. No admission is necessarily intended, nor should be construed, that any of the preceding information constitutes prior art against the present invention.

### SUMMARY

An object of the present application is to provide a process for synthesizing squalane and squalane derivatives. In accordance with an aspect of the present application, there is provided a process for producing a cosmetic, personal care or pharmaceutical composition comprising the steps of: (a) combining a Cₙ-fatty acid and a Cₘ-fatty acid with a solvent to form an electrolysis reaction mixture, wherein the sum of n+m is 32; and performing a Kolbe electrolysis on the electrolysis reaction mixture; (b) subjecting the product of step (a) to a hydroisomerization reaction to produce a C30 saturated, branched hydrocarbon or a mixture of C30 saturated, branched hydrocarbons; and (c) formulating the C30 saturated, branched hydrocarbon with one or more ingredients to produce the cosmetic, personal care or pharmaceutical composition.

In accordance with another aspect, there is provided a cosmetic, personal care or pharmaceutical composition produced by a process for producing a cosmetic, personal care or pharmaceutical composition comprising the steps of: (a) combining a Cₙ-fatty acid and a Cₘ-fatty acid with a solvent to form an electrolysis reaction mixture, wherein the sum of n+m is 32; and performing a Kolbe electrolysis on the electrolysis reaction mixture; (b) subjecting the product of step (a) to a hydroisomerization reaction to produce a C30 saturated, branched hydrocarbon or a mixture of C30 saturated, branched hydrocarbons; and (c) formulating the C30 saturated, branched hydrocarbon with one or more ingredients to produce the cosmetic, personal care or pharmaceutical composition.

### BRIEF DESCRIPTION OF FIGURES

For a better understanding of the application as described herein, as well as other aspects and further features thereof, reference is made to the following description which is to be used in conjunction with the accompanying drawings, where:

FIG. 1 depicts overlaid gas chromatograms of a C30 alkane blend (produced from palmitic fatty acid only), a C30-C34 blend (a heavier emollient produced from a combination of palmitic and steric fatty acids), and a commercially available olive squalane; and

FIG. 2 depicts a spider diagram summarizing the results of sensory characteristic testing.

### DETAILED DESCRIPTION

Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

The present inventors have found that squalane, squalane derivatives and mixtures thereof can be produced using a process that comprises a Kolbe electrolysis step and a subsequent hydroisomerization step. As used herein, the term “squalane derivative” refers to a C30 branched alkane. The squalane or squalane derivative, or combinations thereof, are then formulated into a cosmetic, personal care or pharmaceutical product.

The process of the present application comprises a decarboxylative dimerization of two fatty acids by Kolbe electrolysis to form a C30 hydrocarbon, which is an alkane or alkene. The C30 hydrocarbon is then subjected to a hydroisomerization step to produce a saturated, and branched C30 hydrocarbon.

In one example, in which C16 (palmitic) fatty acid was used as the starting material, a pure stream of C30 alkane (triacontane) is produced. Subsequent hydroisomerization of the

C30 alkane, produces branched, saturated hydrocarbon like squalane. While not exactly the stero-chemical conformation as squalane, the sensory experience of this product will nonetheless be similar. Palmitic fatty acid can be readily purchased from oleo-chemical suppliers.

Kolbe electrolysis

The Kolbe electrolysis reaction (H. Kolbe, Liebigs Ann. Chem. 1849, 69, 257-294) is a chemical reaction process for the decarboxylation of carboxylic acids in processes of making hydrocarbons. This reaction can be used to electrochemically oxidize carboxylic acids to produce alkanes, alkenes, alkane-containing products, alkene-containing products (i.e., compounds that comprise alkanes and alkene, respectively, such as substituted alkanes and substituted alkenes, generated from the Kolbe electrolysis reaction) and mixtures thereof. The reaction proceeds through radical intermediates to yield products based on dimerization of these radicals, such that an Cₙ-acid will combine with an Cₘ-acid to form an alkane and/or alkene comprising m+n-2 carbons along with two carbon dioxide molecules and one hydrogen molecule. The radical intermediates can also lead to shorter alkane and/or alkene products by disproportionation. In the Kolbe electrolysis, only the carboxyl groups participate in the reaction and any unsaturation that may be present in the fatty acid chain is preserved in the reaction product.

The Kolbe electrolysis reaction process may use a single carboxylic acid (in which case the Cₙ-fatty acid and a Cₘ-fatty acid are the same) or a mixture of carboxylic acids (in which case the Cₙ-fatty acid and a Cₘ-fatty acid are different from one another). When a mixture of carboxylic acids is used, the mixture comprises a Cₙ-fatty acid and a Cₘ-fatty acid, where the sum of n+m is 32.

In one embodiment of the process of the present application the Kolbe electrolysis step comprises: combining a Cₙ-fatty acid and a Cₘ-fatty acid with a solvent to form an electrolysis reaction mixture, wherein the sum of n+m is 32; and performing a Kolbe electrolysis on the electrolysis reaction mixture.

Fatty acids that are useful in the process of the present application can be saturated (i.e., do not contain any double bonds) or unsaturated (i.e., containing one or more alkenyl functional groups along the fatty acid chain). Common examples of suitable saturated fatty acids are:
Butyric (butanoic acid): CH₃(CH₂)₂COOH or C4:0;
Caproic (hexanoic acid): CH₃(CH₂)₄COOH or C6:0;
Caprylic (octanoic acid): CH₃(CH₂)₆COOH or C8:0;
Capric (decanoic acid): CH₃(CH₂)₈COOH or C10:0;
Lauric (dodecanoic acid): CH₃(CH₂)₁₀COOH or C12:0;
Myristic (tetradecanoic acid): CH₃(CH₂)₁₂COOH or C14:0;
Palmitic (hexadecanoic acid): CH₃(CH₂)₁₄COOH or C16:0;
Stearic (octadecanoic acid): CH₃(CH₂)₁₆COOH or C18:0;
Arachidic (eicosanoic acid): CH₃(CH₂)₁₈COOH or C20:0; and
Behenic (docosanoic acid): CH₃(CH₂)₂₀COOH or C22:0.

Common examples of suitable unsaturated fatty acids are:
Oleic acid: CH₃(CH₂)₇CH=CH(CH₂)₇COOH or cis-Δ⁹ C18:1
Linoleic acid: CH₃(CH₂)₄CH—CHCH₂CH—CH(CH₂)₇COOH or C18:2
Alpha-linolenic acid: CH₃CH₂CH=CHCH₂CH=CHCH2CH=CH(CH₂)₇COOH or C18:3
Arachidonic acid CH₃(CH₂)₄CH=CHCH₂CH=CHCH₂CH=CHCH₂CH=CH(CH₂)₃COOH or C20:4
Eicosapentaenoic acid or C20:5
Docosahexaenoic acid or C22:6
Erucic acid: CH₃(CH₂)₇CH=CH(CH₂)₁₁COOH or C22:1

As used herein the nomenclature “Cx:y” is used to define fatty acids in terms of the number of carbon atoms in the fatty acid; where x is the number of carbon atoms in the fatty acid chain and y is the number of double bonds. When y is 0, the fatty acid is a saturated fatty acid. The terminology “Cx” is used herein to refer to both saturated and unsaturated fatty acids having x carbon atoms in the fatty acid chain.

By way of non-limiting examples, the Kolbe electrolysis can be performed using a mixture of a C4 fatty acid and a C28 fatty acid, a C5 fatty acid and a C27 fatty acid, a C6 fatty acid and a C26 fatty acid, a C7 fatty acid and a C25 fatty acid, a C8 fatty acid and a C24 fatty acid, a C9 fatty acid and a C23 fatty acid, a C10 fatty acid and a C22 fatty acid, or a mixture of a C11 fatty acid and a C21 fatty acid, a C12 fatty acid and a C20 fatty acid, a C13 fatty acid and a C19 fatty acid, a C14 fatty acid and a C18 fatty acid, or a C15 fatty acid and a C17 fatty acid. These fatty acids can be saturated or unsaturated, or any combination thereof

In another embodiment of the process of the present application, the Kolbe electrolysis is performed using a C16 fatty acid. The C16 fatty acid can be a saturated or an unsaturated fatty acid, or it can be a mixture of C16 fatty acids.

Fatty acids from any feedstock are distilled such that required fatty acids are extracted from the other fatty acids present in the feedstock. For example, for a high yield of C16 fatty acid, palm and palm olein are good sources. However, C16 from other feedstock materials can also be used. The major product of a Kolbe electrolysis of C16 is C30 molecules.

A significant renewable source of the fatty acids comes from the hydrolysis of triglycerides of plant oils and animal fats. The nominal composition of fatty acids from various plant oils and animal fats is given in Table 1.

TABLE 1
Fatty acid composition of selected fats and oils

| Oil or | | | | | | | | | | | | | | | | | |
|---|---|---|---|---|---|---|---|---|---|---|---|---|---|---|---|---|---|
| Fat Source | C2:0 | C10:0 | C12:0 | C14:0 | C16:0 | C16:1 | C18:0 | C18:1 | C18:2 | C18:3 | C20:0 | C20:1 | C22:0 | C22:1 | C24:0 | C24:1 | C26:0 |
| Camelina | | | | 3.4 | 7.8 | | 3.0 | 16.7 | 23.1 | 31.2 | | 12.0 | | 2.8 | | | |
| Canola | | | | | 4.0 | | 2.0 | 62.0 | 22.0 | 10.0 | | | | | | | |
| Carinata | | | | | | | 1.0 | 9.0 | 16.0 | 14.0 | 7.0 | | | 40.0 | | 3.0 | |
| Coconut | | 14.0 | 47.0 | 18.0 | 9.0 | | 3.0 | 6.0 | 2.0 | | | | | | | | |
| Distiler's | | | | | 11.0 | | 2.0 | 28.0 | 58.0 | 1.0 | | | | | | | |
| Corn Ot | | | | | | | | | | | | | | | | | |
| Catropha | | | | 0.1 | 14.2 | 0.7 | 7.0 | 44.7 | 32.8 | 0.3 | 0.2 | | | | | | |
| Lard | | | | 2.0 | 26.0 | | 14.0 | 44.0 | 10.0 | | | | | | | | |
| Palm | | | | 1.0 | 45.0 | | 4.0 | 40.0 | 10.0 | | | | | | | | |
| Palm Kerne | | 4.0 | 48.0 | 16.0 | 8.0 | | 3.0 | 15.0 | 2.0 | | | | | | | | |
| Palm Olein | | | | 1.0 | 37.0 | | 4.0 | 46.0 | 11.0 | | | | | | | | |
| Peanut | | | | | 11.0 | | 2.0 | 48.0 | 32.0 | | | | | | | | |
| Pongamia | | | | | 5.8 | | 5.7 | 57.9 | 14.6 | 2.6 | 3.45 | 10.95 | 4.85 | | 2.3 | | |
| Soybean | | | | | 11.0 | | 4.0 | 24.0 | 54.0 | 7.0 | | | | | | | |
| Sunflower | | | | | 7.0 | | 5.0 | 19.0 | 68.0 | 1.0 | | | | | | | |
| Ta low | | | | 3.0 | 26.0 | | 23.0 | 44.0 | 3.0 | 1.0 | | | | | | | |
| Saipoi Oil | | | | | 3.2 | 0.21 | 1.21 | 13.3 | 16.9 | 12.22 | 0.8 | 9.6 | 0.1 | 40.26 | 0.61 | 2.04 | 0.1 |

Fatty acids useful in the Kolbe electrolysis can also be produced from triglyceride hydrolysis, such as acid- or base- or enzyme-catalyzed hydrolysis. Products of the hydrolysis reaction which can be present in the Kolbe electrolysis reaction solution may include some unreacted triglycerides, diglycerides, monoglycerides, or glycerol depending upon the feedstock. In the present invention preferably the hydrolysis reaction includes a significant aqueous phase and is designed so that all of the feedstock fats and oils are hydrolyzed into a water-insoluble free fatty acids phase which floats on top of the aqueous phase. Preferably the glycerol byproduct of the hydrolysis reaction fully dissolves into the aqueous phase. It may be advantageous to retain or recover the solvent and/or base from the hydrolysis reaction, for the Kolbe electrolysis reaction.

Suitable solvents for the Kolbe electrolysis include, for example, C1-C3 alcohols. For example, the solvent employed in the Kolbe electrolysis reaction is methanol or ethanol or a mixture of C1-C3 alcohols. The Kolbe electrolysis reaction is tolerant to the presence of water, and water may be present in this reaction in amounts up to 40% by volume. In certain embodiments, solubility of reaction components and electrical conductivity of electrolysis solution may be improved in a solvent system which comprises a mixture of alcohol and water. Accordingly, in some embodiments, the solvent comprises water at an amount of about 2% to about 50%, about 5% to about 45%, about 10% to about 40% or about 20% to about 30% by volume (e.g., water in ethanol).

The initial reaction mixture for the Kolbe electrolysis reaction may not be a solution (with the feedstock and other components dissolved) at ambient temperature (22° C.). During the Kolbe electrolysis reaction, the neutralized (i.e., salt) form of the fatty acid must be in solution. The free fatty acid can exist as a separate phase. As the carboxylate ion form of the fatty acid is converted to hydrocarbon during electrolysis, the base, which is formed in this reaction, reacts with the free fatty acid to form a salt, thereby drawing more fatty acid (in its salt form) into solution. This continues until all the fatty acid is consumed. In one embodiment, the fatty acid is continuously supplied to the reaction, and hydrocarbon product is continuously removed, which helps maintaining a constant reaction rate, and performing Kolbe electrolysis in steady-state mode.

In some embodiments of the present invention, the Kolbe electrolysis can be performed at temperatures below or above room temperature. The Kolbe electrolysis reaction is conducted at a temperature in the range of about 0° C. to about 100° C., or of about 0° C. to about 80° C., or of about 15° C. to about 80° C. Higher pressures than atmospheric pressure can be employed to prevent loss of the solvent or the boiling over of the reaction mixture.

In the Kolbe electrolysis reaction, a base can be added to partially convert the carboxylic acid group of the fatty acids to a carboxylate salt prior to initiating or during the Kolbe reaction undergoing electrolysis. In some embodiments the fatty acids will be neutralized by ranges from about 1 to 80, 1 to 60, or 1 to 25 percent. In this case the percent means the concentration of the neutralized fatty acid in molar units relative to the total carboxylic acid molar concentration. Suitable bases for neutralization of the fatty acids are hydroxide, alkoxide or carbonate salts of sodium or potassium. Amine bases can also be used.

In some embodiments an electrolyte can be added to the Kolbe reaction mixture to increase the Kolbe reaction mixture electrical conductivity. Suitably an electrolyte to improve the Kolbe reaction mixture electrical conductivity can be selected from the group consisting of perchlorate, p-toluenesulfonate or tetrafluoroborate salts of sodium or tetraalkylammonium or a mixture thereof. Anions other than from these electrolytes, or other than the carboxylates of the substrate carboxylic acids, may interfere and should not be present. An increase in mixture conductivity corresponds with a decrease in mixture resistivity.

The material of the cathode in Kolbe electrolysis is usually stainless steel, nickel, or graphite, although other suitable materials can also be used, including platinum or gold. The material of the anode is typically platinum, at least at the reacting surface of the anode. The anode can be a foil or plate consisting of the anode material or the anode material can be plated on or affixed to a support material such as titanium, niobium, graphite, or glass, with the preferred support material being titanium or niobium. For example, an anode consisting of a 1 mm-thick titanium plate electroplated with 1 micrometer of platinum can be used for the Kolbe electrolysis to give a productivity value approximately equivalent to that found using a platinum foil anode. Other materials can also be used as the anode, including non-porous graphite, gold or palladium.

The current density, defined as the current supplied to the electrode divided by the active surface area of the electrode, applied to the Kolbe electrolysis can be in the range of about 10 to about 1000 mA/cm².

In some embodiments of the present invention when unsaturated fatty acids are parts of fatty acid mixture, acetic acid is added to lower a passivation voltage in Kolbe electrolysis, for example, as described in International PCT application WO 2016/0080335. The amount of acetic acid is between about 0.2 weight percent to about 20 weight percent of the total carboxylic acid.

The hydrocarbon product of the Kolbe electrolysis can be separated from the reaction mixture using a reaction product separator, wherein the reaction product separator is any device that can separate liquid or solid reaction product from liquid solution. Examples of reaction product separators include, but not limited to, a centrifugal separator, a cyclone, a gravity-driven separator, a settling tank, a filtration system and a distillation system.

Hydroisomerization

The next step of the process is a hydroisomerization of the C30 hydrocarbon product of the Kolbe electrolysis reaction to modify the properties of the hydrocarbon such that it is more suitable for use in cosmetic, personal care or pharmaceutical compositions.

The hydroisomerization reaction is performed in the presence of hydrogen gas and a catalyst having a metal component to catalyze skeletal isomerization, yielding saturated, branched hydrocarbons having the same molecular weight as the starting C30 hydrocarbon product of the Kolbe electrolysis step. The resulting hydrocarbon material is more stable to oxidation and is more fluid at lower temperatures, which are desirable properties.

In an example of a hydroisomerization reaction, the catalyst is a silica/alumina-based zeolite containing impregnated platinum group metals, the reaction temperature is between about 250° C. to about 400° C. or about 275° C. to about 400° C., the reaction pressure is between about 10 bar to about 400 bar or from about 10 bar to about 100 bar, and the hydrogen gas to a hydrocarbon ratio is about 100 to about 1000 or about 400 to about 1000.

A side reaction of the hydroisomerization process is a hydrocarbon cracking, which produces short-chain hydrocarbons. The crude product of the hydroisomerization is a mixture of squalane and squalane derivatives (long-chain hydrocarbons) and short-chain hydrocarbons with a carbon number of less than 30. The squalane and squalane derivatives are separated from short-chain hydrocarbons by distillation or any other separation technique known to those knowledgable in the art, such that the final product is a blend of C30 alkanes that can include any combination of one or more squalane derivatives with or without squalane.

Formulation

The product of the hydroisomerization step is subsequently formulated in to a cosmetic, personal care or pharmaceutical composition according to routine methods well known in the art. The cosmetic, personal care or pharmaceutical composition can comprise one or more excipients, diluents or active ingredients as would be readily determined by the person skilled in formulation methodology, based on the end use of the composition.

To gain a better understanding of the invention described herein, the following examples are set forth. It should be understood that these examples are for illustrative purposes only. Therefore, they should not limit the scope of this invention in any way.

EXAMPLES

EXAMPLE 1:

Kolbe electrolysis was performed using a feedstock blend of palmitic and steric acids to produce a blend of C30 -C34 hydrocarbons. A second Kolbe electrolysis was performed using a feedstock of palmitic acid alone to produce a blend of C30 hydrocarbons. The resultant hydrocarbons were each subjected to hydroisomerization using a silica/alumina-based zeolite containing impregnated platinum as the catalyst at a reaction temperature of between 275° C. and 400° C., a reaction pressure of between 10 bar and 100 bar. The hydrogen gas to hydrocarbon ratio in each case was between 400 and 1000. The alkane products were purified by removal of the short-chain hydrocarbon products of the cracking side reaction by distillation.

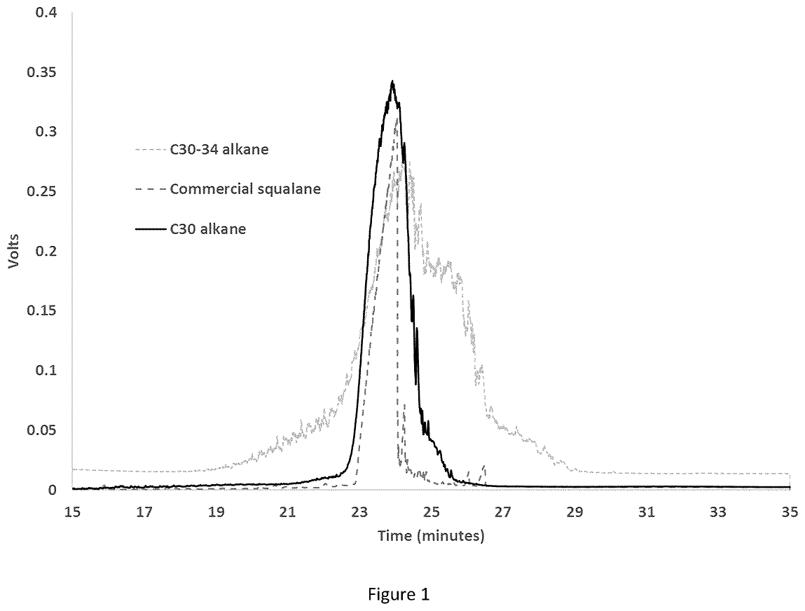

FIG. 1 shows a comparison of the gas chromatograms for the C30 alkane blend (produced from palmitic fatty acid only), the C30-C34 blend (a heavier emollient produced from a combination of palmitic and steric fatty acids), and commercially available olive squalane. The blend made from the palmitic acid feedstock more closely matched squalane than did the blend made from a combination of palmitic and stearic acid, thereby confirming that the process generated a blend of alkanes that closely approximates squalane.

The sensory characteristics of hydroisomerized materials comprising the blends (i) the C30-C34 alkanes; and (ii) the C30 alkanes were compared to commercially purchased squalane. The methods of testing are given in the Table below.

| Sensory | | | |
|---|---|---|---|
| Property | Description | Test | Results |
| Smoothness | Texture of product | One drop of product was placed on | All three products were |
| | during application | inner forearm and applied to skin in | equally smooth during |
| | | 1 × 1″ area. The texture was tested to | application. |
| | | determine if product felt smooth | |
| | | while applying the product or if | |
| | | application caused friction or drag | |
| | | on the skin. | |
| Glossiness | Degree of light | One drop of each product was | The C30 blend and |
| | reflected off skin | placed on inner forearm of subject | Squalane were equally |
| | | and the degree of light reflected was | glossy, whereas the C30- |
| | | compared amongst the three | C34 blend was less |
| | | samples. | glossy. |
| Absorbency/ | The amount of time | One drop of product was placed on | The C30-C34 blend took |
| Playtime | required for product | inner forearm and applied to skin in | the least time to absorb |
| | to absorb into skin | 1 × 1″ area and was timed to measure | into skin, while the |
| | during application | how long the product takes to | Squalane required the |
| | | absorb into skin. | most time. |
| Oiliness | How wet the | One drop of product was placed on | The C30-C34 blend felt |
| | product feels during | inner forearm and applied to skin in | the most wet on the |
| | application | 1 × 1″ area. The texture was tested to | skin during application, |
| | | determine if the product felt wet or | while Squalane felt the |
| | | dry during application. | least wet. |
| Spreadability | Ease of moving | One drop of each product was | The Squalane droplet |
| | product | placed on a flat piece of paper, the | travelled the furthest |
| | | paper was then held at a 45° angle | distance, while the C30- |
| | | for a set period of time. The | C34 blend travelled the |
| | | distance the oil droplets travelled by | shortest. |
| | | gravity determined the ability to | |
| | | spread the product. | |

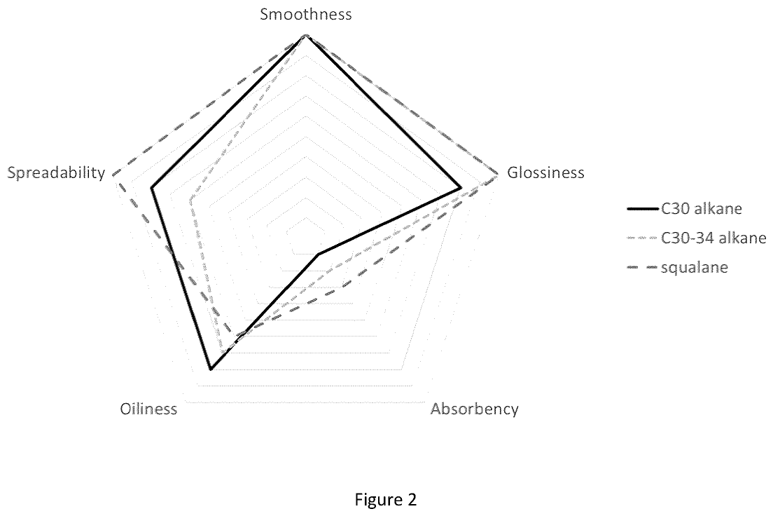

The results of this testing are shown qualitatively in the spider diagram of FIG. 2. The results demonstrate that the C30 blend was surprisingly similar in sensory characteristics to the commercial squalane, more so than the C30 -C34 blend, which would have been expected to have more similar characteristics based on the narrow size range in the blend. Accordingly, the C30 blend produced according to the process described herein can be used as a synthetic substitute for squalane extracted from natural sources.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A process for producing a cosmetic, personal care or pharmaceutical composition comprising the steps of:
(a) combining a Cₙ-fatty acid and a Cₘ-fatty acid with a solvent to form an electrolysis reaction mixture, wherein the sum of n+m is 32; and performing a Kolbe electrolysis on the electrolysis reaction mixture;
(b) subjecting the product of step (a) to a hydroisomerization reaction to produce a C30 saturated, branched hydrocarbon or a mixture of C30 saturated, branched hydrocarbons; and
(c) formulating the C30 saturated, branched hydrocarbon with one or more ingredients to produce the cosmetic, personal care or pharmaceutical composition.

2. The process of claim 1, wherein the Cₙ-fatty acid and Cₘ-fatty acid are both a C16 fatty acid.

3. The process of claim 1, wherein the Cₙ-fatty acid is a C8 fatty acid and the Cₘ-fatty acid is a C24 fatty acid, the Cₙ-fatty acid is a C9 fatty acid and the Cₘ-fatty acid is a C23 fatty acid, the Cₙ-fatty acid is a C10 fatty acid and the Cₘ-fatty acid is a C22 fatty acid, the Cₙ-fatty acid is a C11 fatty acid and the Cₘ-fatty acid is a C21 fatty acid, the Cₙ-fatty acid is a C12 fatty acid and the Cₘ-fatty acid is a C20 fatty acid, the Cₙ-fatty acid is a C13 fatty acid and the Cₘ-fatty acid is a C19 fatty acid, the Cₙ-fatty acid is a C14 fatty acid and the Cₘ-fatty acid is a C18 fatty acid, or the Cₙ-fatty acid is a C15 fatty acid and the Cₘ-fatty acid is a C17 fatty acid.

4. The process of claim 1, wherein in step (a) from 1 -25 mol % of the fatty acids are neutralized with a base.

5. The process of claim 1, wherein step (a) is carried out at a current density of from about 10 to about 1000 mA/cm² and at a temperature of from about 0° C. to about 80° C.

6. The process of claim 1, wherein the hydroisomerization of step (b) is performed in the presence of hydrogen gas and a catalyst.

7. The process of claim 6, wherein the catalyst is a silica/alumina-based zeolite containing impregnated platinum.

8. The process of claim 1, wherein the hydroisomerization reaction temperature is between about 275° C. to about 400° C.

9. The process of claim 1, wherein the hydroisomerization reaction pressure is between about 10 bar to about 100 bar.

10. The process of claim 1, wherein in the hydroisomerization reaction, the the hydrogen gas to a hydrocarbon ratio is about 400 to about 1000.

11. A cosmetic, personal care or pharmaceutical composition produced by the process of claim 1.
